(19) United States
(12) Patent Application Publication
(10) Pub. No.: US 2006/0247569 A1
(43) Pub. Date: Nov. 2, 2006

(54) Implantable cerebral spinal fluid flow device and method of controlling flow of cerebral spinal fluid

(75) Inventors: William J. Bertrand, Ventura, CA (US); William Sugleris, Goleta, CA (US)

Correspondence Address: IPLM GROUP, P.A., POST OFFICE BOX 18455, MINNEAPOLIS, MN, 55418, US

(73) Assignee: Medtronic, Inc.

(21) Appl. No.: 11/118,766

(22) Filed: Apr. 29, 2005

(51) Int. Cl. A61M 5/00

(52) U.S. Cl. 604/9

(57) ABSTRACT

An implantable cerebral spinal fluid flow device and method. A body has an inlet for the cerebral spinal fluid, an outlet for the cerebral spinal fluid and a first interior cavity fluidly coupled with the inlet and the outlet. A first rotational element is mounted in the first interior cavity in a pathway between the inlet and the outlet. The first rotational element provides resistance to flow of the cerebral spinal fluid from the inlet to the outlet. In the method, the cerebral spinal fluid flow device is implanted. Resistance to rotation of the first rotational element to flow of the cerebral spinal fluid from the inlet to the outlet is provided.

### FIELD OF THE INVENTION

This invention relates generally to implantable fluid flow control devices and methods and, more particularly, to such devices and methods for controlling flow of cerebral spinal fluid.

### BACKGROUND OF THE INVENTION

Ventricles of the brain contain cerebrospinal fluid which cushions the brain against shock. Cerebral spinal fluid is constantly being secreted and absorbed by the body usually in equilibrium. Cerebral spinal fluid is produced in the ventricles of the brain, where under normal conditions, it is circulated in the subarachnoid space and reabsorbed into the bloodstream, predominantly via the arachnoids villi attached to the superior sagittal sinus. However, if blockages in circulation of cerebral spinal fluid, perhaps in the ventricles, cerebral spinal fluid can't be reabsorbed by the body at the proper rate.

This can create a condition known as hydrocephalus which is a condition marked by an excessive accumulation of fluid violating the cerebral ventricles, then the brain and causing a separation of the cranial bones. Hydrocephalus is a condition characterized by abnormal flow, absorption or formation of cerebrospinal fluid within the ventricles of the brain which subsequently increases the volume and pressure of the intracranial cavity. If left untreated, the increased intracranial pressure can lead to neurological damage and may result in death.

Over the past 40 years, a common treatment for hydrocephalus patients has been the cerebrospinal fluid shunt. The standard shunt consists of the ventricular catheter, a valve and a distal catheter. The excess cerebrospinal fluid is typically drained from the ventricles to a suitable cavity, most often the peritoneum or the atrium. A catheter is tunneled into the brain through a burr hole in the skull. The catheter is placed into ventricles to shunt cerebral spinal fluid to other areas of the body, principally the peritoneum, where it can be reabsorbed. The presence of the shunt relieves pressure from cerebral spinal fluid on the brain.

A flow or pressure regulating valve is usually placed along the catheter path. Differences in pressure due, at least in part, to differences in vertical position between the inlet (ventricles) and the outlet (peritoneum) can create too much drainage with such a flow or pressure regulating valve.

Current shunt valves are primarily pressure relief designs with a predetermined or adjustable opening pressure. Once open, flow rate is essentially unrestricted and over drainage can occur. Over drainage can lead to slit ventricles, slit ventricle syndrome, loss of brain compliance, shunt occlusion, sub-dural hematoma and many other complications.

Some shunt valves are also prone to becoming clogged. A clogged shunt valve could result in serious complications through a failure to provide proper drainage of cerebral spinal fluid from the ventricles of the brain.

Thus, there is needed a device and a method for reliably providing a controlled fluid flow for a cerebral spinal shunt, namely a cerebral spinal fluid control device and method which reliably provides shunting of cerebral spinal fluid without clogging and which provides a controlled flow rate preventing over drainage.

Another problem facing a medical professional when dealing with cerebral spinal fluid shunting devices and methods is determining whether or not the shunt is working, i.e., whether the shunt is still providing cerebral spinal fluid drainage and, possibly, at what flow rate. If the device is not working properly, the surgeon generally performs a shunt revision in which the shunting device is either revised or replaced. However, such a shunt revision is an invasive procedure that should be avoided if the procedure is not necessary.

One technique for determining if cerebral spinal fluid flow is present involves the injection of a contrast agent into the cerebral spinal fluid system following by several imaging sessions to monitor clearance of the contrast media, a procedure generally referred to as “shuntogram.” The procedure is generally time consuming, invasive and expensive. Further, if a patient is in critical condition, time may not be available to allow for the performance of a “shuntogram.”

### BRIEF SUMMARY OF THE INVENTION

A reliable device and method for cerebral spinal fluid shunting from the ventricles of the brain alleviates both the problem over drainage due to lack of fluid flow control as well as the problem of reliability. In an embodiment, a rotational element, preferably a pinwheel valve, avoids the use of tiny, restrictive or tortuous passages to control cerebral spinal fluid flow that can become clogged with debris. A braking mechanism can be associated with the rotational element to provide control of cerebral spinal fluid flow.

In another embodiment, a remotely detectable element, such as a magnetic element, can be affixed to the rotational element, e.g., pinwheel, in the fluid flow control device, and sensing movement, e.g., rotation, of the element non-invasively. Qualitatively, movement or rotation of the element can immediately and easily determine whether or not the fluid flow control device operational, i.e., shunting cerebral spinal fluid, or whether the fluid flow control device has become clogged or otherwise malfunctioned.

Further, a quantitative measurement of the amount of flow of cerebral spinal fluid can easily be obtained by measuring the rotational speed of the rotational element, e.g., pinwheel, and performing a simple arithmetic calculation. A quantitative measurement can be important because the current flow rate can be compared with a baseline of flow rate established at or near the time of implantation, or another prior time, to possibly predict impending shunt failure. The potential ability to predict shunt failure could allow safer, non-emergency revisions and result in less neurological deficit and/or injury to patients.

In an embodiment, the present invention provides an implantable cerebral spinal fluid flow device. A body has an inlet for the cerebral spinal fluid, an outlet for the cerebral spinal fluid and a first interior cavity fluidly coupled with the inlet and the outlet. A first rotational element is mounted in the first interior cavity in a pathway between the inlet and the outlet. The first rotational element provides resistance to flow of the cerebral spinal fluid from the inlet to the outlet.

In a preferred embodiment, the first rotational element comprises a pinwheel mounted in the first interior cavity.

In a preferred embodiment, the pinwheel is braked to provide the resistance to flow of the cerebral spinal fluid.

In a preferred embodiment, the device further has a pre-loaded spring mounted against the pinwheel to provide braking.

In a preferred embodiment, the pre-loaded spring is mounted radially with respect to the pinwheel to provide a resistance to rotation of the pinwheel.

In a preferred embodiment, the pre-loading of the pre-loaded spring is adjustable to provide a variable pressure against the pinwheel.

In a preferred embodiment, a second rotational element, rotationally coupled with the first rotational element, is mounted in a second interior cavity and a viscous fluid is contained in the second interior cavity providing rotational resistance to the second rotational element.

In a preferred embodiment, the second rotational element is mounted co-axially with the first rotational element.

In a preferred embodiment, the second rotational element is a pinwheel.

In a preferred embodiment, the pinwheel has a plurality of blades and the each of the plurality of blades contains at least one hole.

In a preferred embodiment, the at least one hole is selected in size to provide a selected resistance to rotation.

In a preferred embodiment, the viscous fluid has a shear that increases with a rotational speed of the second rotational element.

In a preferred embodiment, the viscous fluid comprises a fluid whose viscosity increases as the shear increases.

In a preferred embodiment, the viscous fluid comprises silicone fluid.

In a preferred embodiment, a remotely detectable element is mounted with respect to the first rotational element.

In a preferred embodiment, the remotely detectable element provides information regarding flow of the cerebrospinal fluid.

In a preferred embodiment, the remotely detectable element provides information regarding a flow rate of the cerebrospinal fluid.

In a preferred embodiment, the remotely detectable element provides information regarding a rotational speed of the first rotational element.

In another embodiment, the present invention provides a method of controlling a flow of cerebral spinal fluid. A cerebral spinal fluid flow device is implanted. The cerebral spinal fluid flow device has an inlet for the cerebral spinal fluid, an outlet for the cerebral spinal fluid and a first interior cavity fluidly coupled with the inlet and the outlet, and a first rotational element mounted in the first interior cavity in a pathway between the inlet and the outlet. Resistance to rotation of the first rotational element to flow of the cerebral spinal fluid from the inlet to the outlet is provided.

In a preferred embodiment, the pinwheel is braked to provide the resistance to rotation.

In a preferred embodiment, resistance to rotation is provided radially with respect to the pinwheel.

In a preferred embodiment, the resistance to rotation is adjustable.

In a preferred embodiment, the resistance to rotation is provided by the body having a second interior cavity, a second rotational element mounted in the second interior cavity; and a viscous fluid contained in the second interior cavity providing rotational resistance to the second rotational element, wherein the second rotational element and the first rotational element are rotationally coupled.

In a preferred embodiment, an element mounted with respect to the first rotational element is remotely detected.

In a preferred embodiment, information regarding the flow of cerebral spinal fluid is remotely detected.

In a preferred embodiment, information regarding a flow rate of the cerebral spinal fluid is remotely detected.

In a preferred embodiment, information regarding a rotational speed of the first rotational element is detected.

In a preferred embodiment, the remotely detectable element is a magnetic element.

In a preferred embodiment, the remotely detectable element comprises a magnetic element.

### BRIEF DESCRIPTION OF THE DRAWINGS

FIG. 1 is a cut-away perspective view of cerebral spinal fluid flow control device implanted into the cranium of a patient;

FIG. 2 is a cross-sectional side view of a cerebral spinal fluid flow control device in accordance with an embodiment of the present invention;

FIG. 3 is a cross-sectional front view of the cerebral spinal fluid flow control device of FIG. 2;

FIG. 4 is a cross-sectional side view of an alternative embodiment of a cerebral spinal fluid flow control device;

FIG. 5 is a cross-sectional front view of the cerebral spinal fluid flow control device of FIG. 4; and

FIG. 6 is a side view of an adjustable spring useful for providing adjustable braking for the cerebral spinal fluid control devices of FIG. 2 through FIG. 5.

### DETAILED DESCRIPTION OF THE INVENTION

Consistent and reliable drainage of cerebral spinal fluid from one area of the body to another, e.g., from a ventricle or ventricles of the brain to another region of the body such as the peritoneum pr sagittal sinus, can be desirable. A consistent and reliable drainage method and system can minimize the expense as well as trauma and inconvenience to the patient associated with cerebral spinal fluid revision surgery and can also lesson risk to the patient due to an inoperative cerebral spinal fluid drainage system.

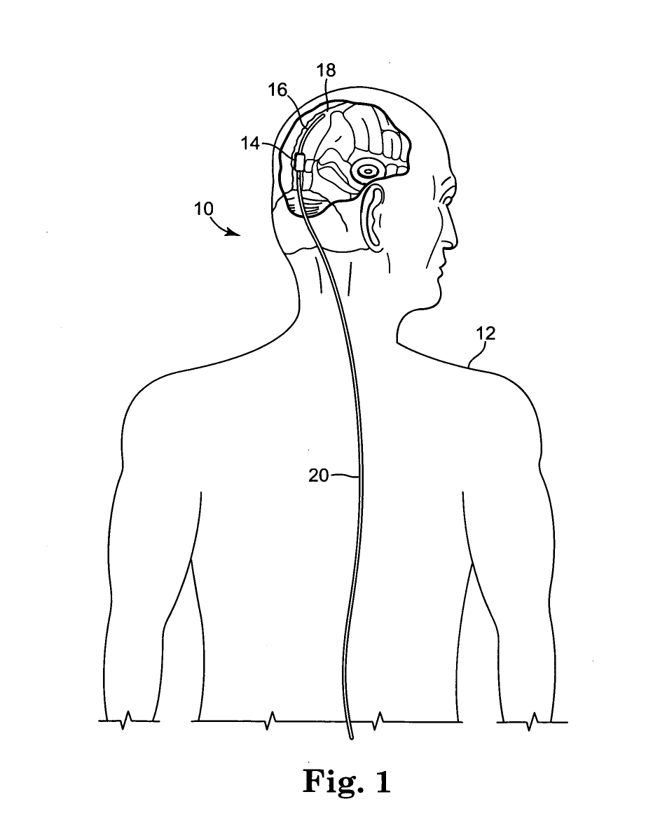

FIG. 1 illustrates an embodiment of a cerebral spinal fluid shunt, or drainage, system 10 for draining cerebral spinal fluid from one area, preferably the ventricles of brain, of the body of patient 12 to another area of the body of patient 12. Cerebral spinal fluid can preferably be drained to the peritoneum and/or atrium and, alternatively, to the sagittal sinus. Shunt system 10 may consist solely of a catheter having a lumen to transport cerebral spinal fluid or may consist, as illustrated in FIG. 1, flow control device 14.

Flow control device 14 may be located anywhere along the path of cerebral spinal fluid flow. For example, flow control device 14 may be located at or near the inlet for cerebral spinal fluid, e.g., at or near the ventricles, or may be located at or near the outlet for the cerebral spinal fluid, e.g., at or near the peritoneum. Alternatively, flow control device 14 may be located as illustrated in FIG. 1 along the flow path between the inlet and outlet. In particular, by way of example, flow control device 14 may be near the cranium 24.

Ventricular catheter 16, having a lumen, is connects flow control device 14 to inlet location 18 in the ventricle of patient 12. It is to be recognized and understood that other locations, other than inlet location 18, can be used. Distal catheter 20 connects flow control device 14 with an outlet for cerebral spinal fluid, not shown, which preferably is in the peritoneum. It is to be recognized and understood that other outlet locations can be used. Examples of other possible outlet locations include the atrium and the sagittal sinus.

Although not required, flow control device 14 can help alleviate cerebral spinal fluid flow differential due to different positioning of the body. For example, when the body is supine, the difference in elevation between the inlet of ventricular catheter 16 and the outlet of distal catheter 20 may be relatively small. Thus, the pressure differential due to elevation between the inlet and outlet may also be relatively small. This may result in a relatively small flow rate of cerebral spinal fluid through shunt system 10.

However, when the body is erect, for example, the difference in elevation between the inlet of ventricular catheter 16 and the outlet of distal catheter 20 may be relatively large. Thus, the pressure differential due to elevation between the inlet and outlet may also be relatively large. This may result in a relatively large flow rate of cerebral spinal fluid through shunt system 10.

The presence of a flow control device 14 in shunt system 10 can help to stabilize the rate of flow of cerebral spinal fluid through shunt system 10 by limiting the higher flow rates associated with, for example, an erect position of the body.

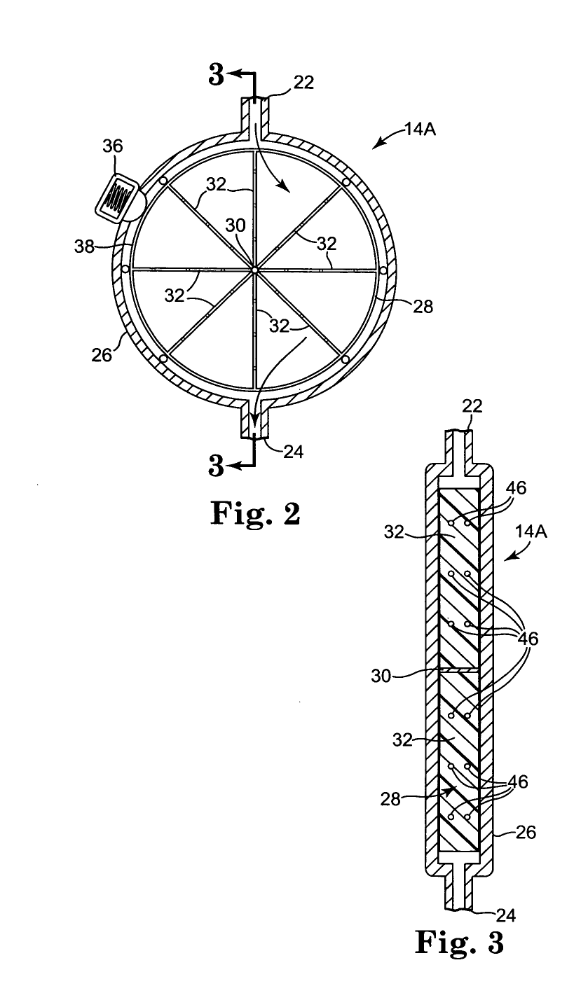

FIG. 2 and FIG. 3 illustrate cross-sectional side and front views, respectively of flow control device 14A of an embodiment of the invention. Ventricular catheter 16 (not shown) is fluidly connected to inlet port 22 of flow control device 14A and distal catheter 20 is fluidly connected to outlet port 24 of flow control device 14A. Thus, cerebral spinal fluid can flow through flow control device 14A from inlet port 22 to outlet port 24, generally in a top to bottom direction as illustrated in FIGS. 2 and 3.

Body 26 of flow control device 14A holds a rotational element, namely pinwheel 28, mounted on axis 30. Pinwheel 28 is rotatable around axis 30. Cerebral spinal fluid enters flow control device 14A through inlet port 22 and is directed around one side of pinwheel 28 (the right hand in FIG. 2). Cerebral spinal fluid impinges on blades 32 forcing pinwheel 28 to rotate in order for a substantial amount of cerebral spinal fluid to pass through flow control device 14A and exit outlet port 24.

Rotational element or pinwheel 28 has a resistance to rotation. That is, rotational element or pinwheel 28 is resistant to the flow of cerebral spinal fluid through flow control device 14A because pinwheel 28 is resistant to rotation.

Such resistance to rotation may be provided by a number of different mechanical or magnetic techniques.

In one alternative embodiment, body 26 of flow control device 14A also contains a viscous fluid, e.g., silicone fluid, 34 which provides drag to the free rotation of pinwheel 28.

Blades 32 of pinwheel 28 may contain holes 46, for example, if viscous fluid were employed as a damping agent, to allow some, but not all, of cerebral spinal fluid to pass through flow control device 14A without or with relatively little resistance.

The number and/or size of holes 46 may be adjusted in order to modify the amount of or proportion of cerebral spinal fluid that is subject to relatively little resistance.

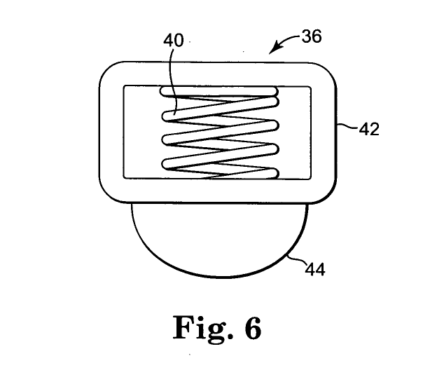

In another alternative embodiment, spring 36 is mounted along the circumference of pinwheel 28 and mechanically impinges against outside rim 38 of pinwheel 28 providing mechanical resistance to the free rotation of pinwheel 28. Spring 36, shown in greater detail in FIG. 6, has coil spring 40 mounted in body 42. Nub 44 is responsive to coil spring 40 and, when mounted in flow control device 14A, is adapted to press against or impinge against outside rim 38 of pinwheel 28. Coil spring 40 may be adjustable, as, for example, by a set screw to adjust the amount of tension or pressure between spring 36 and pinwheel 28.

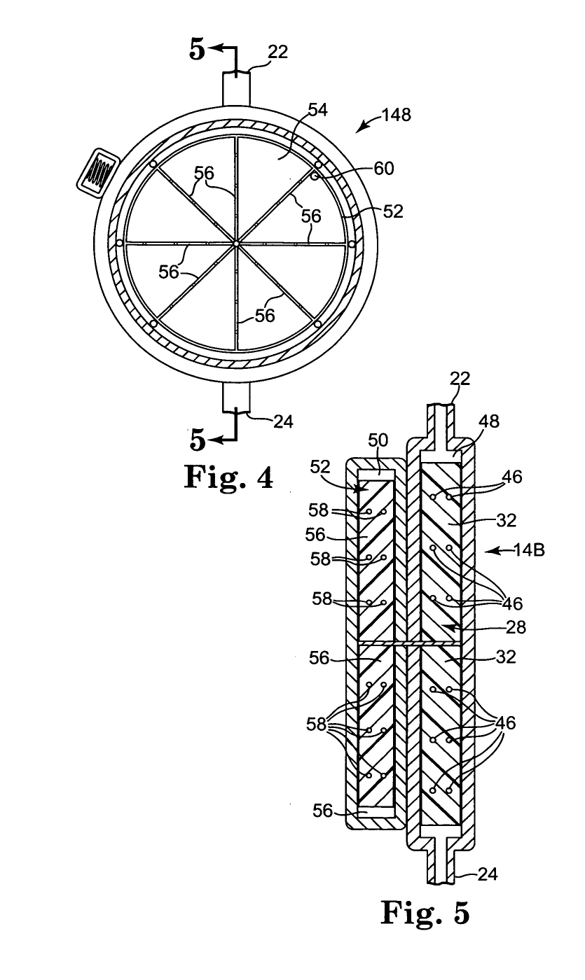

FIG. 4 and FIG. 5 illustrate cross-sectional side and front views, respectively of flow control device 14B of an alternative embodiment of the invention. Ventricular catheter 16 (not shown) is fluidly connected to inlet port 22 of flow control device 14B and distal catheter 20 is fluidly connected to outlet port 24 of flow control device 14B. Thus, cerebral spinal fluid can flow through flow control device 14B from inlet port 22 to outlet port 24, generally in a top to bottom direction as illustrated in FIGS. 4 and 5.

Body 26 of flow control device 14B has two interior cavities. First interior cavity 48 holds a first rotational element, namely pinwheel 28, mounted on axis 30. Pinwheel 28 is rotatable around axis 30. Cerebral spinal fluid enters first interior cavity 48 of flow control device 14B through inlet port 22 and is directed around one side of pinwheel 28 (the right hand in FIG. 4). Cerebral spinal fluid impinges on blades 32 forcing pinwheel 28 to rotate in order for a substantial amount of cerebral spinal fluid to pass through flow control device 14B and exit outlet port 24.

Second interior cavity 50 holds a second rotational element, namely pinwheel 52, mounted co-axially with respect to pinwheel 28. Second interior cavity 50 contains a viscous fluid 56, e.g., silicone fluid. Blades 58, fixed to axle 30, rotate through viscous fluid 56 providing a resistance to rotation of pinwheel 52. Since pinwheel 52 and pinwheel 28 are co-axial, a resistance to rotation provided to pinwheel 52 will provide a resistance to rotation to pinwheel 28 which, in turn, will provide a resistance to rotation of cerebral spinal fluid passing through flow control device 14B.

While one particular mechanism has been illustrated for providing resistance to rotation of one rotational element, e.g., pinwheel 28, by providing resistance to rotation to a second rotational element, e.g., pinwheel 52, it should be recognized and understood that other mechanical and magnetic relationships between the two rotational elements are possible and are contemplated. For example, it is not necessary that the two rotational elements be co-axial, only that resistance to rotation of one element provides some resistance to rotation of the other element.

Blades 56 of pinwheel 52 may contain holes 58 to allow some, but not all, of viscous fluid to pass through blades 56 without or with relatively little resistance. The number and/or size of holes 58 may be adjusted in order to modify the amount of or proportion of viscous fluid that is subject to relatively little resistance. This adjustment will modify the amount of resistance of pinwheel 52 to rotation.

Magnet 60 may be mounted anywhere on either pinwheel 28 or pinwheel 52. As either pinwheel 28 rotates, magnet 60 will also rotate. Note that even if magnet 60 is mounted exactly co-axially with respect to either pinwheel 28 or pinwheel 52, that magnet 60 will still rotate. The rotation of magnet 60 may transcutaneously sensed by well know and standard magnetic measuring equipment. The rotation of magnet 60 is directly proportional to the speed of rotation of pinwheel 28. The speed of rotation of pinwheel 28 is directly proportional to the rate of flow of cerebral spinal fluid through flow control device 14B. Thus, the presence of magnet 60 allows the non-invasive determination of whether or not cerebral spinal fluid is flowing through flow control device 14B, whether magnet 60 is rotating at all, and will also allow a determination of the amount of flow of cerebral spinal fluid through flow control device 14B, using the speed of rotation of magnet 60 using well know mathematical techniques.

While magnet 60 has been described with respect to flow control device 14B, it is to be recognized and understood that magnet 60 could also be utilized equally well with respect to flow control device 14A by mounting magnet 60 with respect to pinwheel 28.

It may be preferable to provide anti-thrombogenic and/or clot busting properties to either flow control device 14A or 14B. Such anti-thrombogenic and/or clot busting properties are described in co-pending U.S. patent application filed on even date herewith in the names of Ari Moskowitz and William J. Bertrand and entitled “Anti-Thrombogenic Venous Shunt System and Method”, carrying attorney docket number 151P21060US01, the contents of which are hereby incorporated by reference.

Thus, embodiments of the implantable cerebral spinal fluid flow device and method of controlling flow of cerebral spinal fluid are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. An implantable cerebral spinal fluid flow device adapted for use with cerebral spinal fluid, comprising:
a body having an inlet for said cerebral spinal fluid, an outlet for said cerebral spinal fluid and a first interior cavity fluidly coupled with said inlet and said outlet; and
a first rotational element mounted in said first interior cavity in a pathway between said inlet and said outlet;
said first rotational element providing resistance to flow of said cerebral spinal fluid from said inlet to said outlet.

2. An implantable cerebral spinal fluid flow device as in claim 1 wherein said first rotational element comprises a pinwheel mounted in said first interior cavity.

3. An implantable cerebral spinal fluid flow device as in claim 2 wherein said pinwheel is braked to provide said resistance to flow of said cerebral spinal fluid.

4. An implantable cerebral spinal fluid flow device as in claim 3 further comprising a pre-loaded spring mounted against said pinwheel to provide braking.

5. An implantable cerebral spinal fluid flow device as in claim 4 wherein said pre-loaded spring is mounted co-axially with respect to said pinwheel to provide a resistance to rotation of said pinwheel.

6. An implantable cerebral spinal fluid flow device as in claim 1 wherein said pre-loading of said pre-loaded spring is adjustable to provide a variable pressure against said pinwheel.

7. An implantable cerebral spinal fluid flow device as in claim 1:
wherein said body has a second interior cavity, said implantable cerebral spinal fluid flow device further comprising:
a second rotational element mounted in said second interior cavity; and
a viscous fluid contained in said second interior cavity providing rotational resistance to said second rotational element;
wherein said second rotational element and said first rotational element are rotationally coupled.

8. An implantable cerebral spinal fluid flow device as in claim 7 wherein said second rotational element is mounted radially with said first rotational element.

9. An implantable cerebral spinal fluid flow device as in claim 7 wherein said second rotational element comprises a pinwheel.

10. An implantable cerebral spinal fluid flow device as in claim 9 wherein said pinwheel has a plurality of blades and wherein said each of said plurality of blades contains at least one hole.

11. An implantable cerebral spinal fluid flow device as in claim 10 wherein said at least one hole is selected in size to provide a selected resistance to rotation.

12. An implantable cerebral spinal fluid flow device as in claim 7 wherein said viscous fluid has a shear that increases with a rotational speed of said second rotational element.

13. An implantable cerebral spinal fluid flow device as in claim 12 wherein said viscous fluid comprises a fluid whose viscosity increases as said shear increases.

14. An implantable cerebral spinal fluid flow device as in claim 12 wherein said viscous fluid comprises silicone fluid.

15. An implantable cerebral spinal fluid flow device as in claim 1 further comprising a remotely detectable element mounted with respect to said first rotational element.

16. An implantable cerebral spinal fluid flow device as in claim 15 wherein said remotely detectable element provides information regarding flow of said cerebrospinal fluid.

17. An implantable cerebral spinal fluid flow device as in claim 15 wherein said remotely detectable element provides information regarding a flow rate of said cerebrospinal fluid.

18. An implantable cerebral spinal fluid flow device as in claim 17 wherein said remotely detectable element provides information regarding a rotational speed of said first rotational element.

19. An implantable cerebral spinal fluid flow device as in claim 15 wherein said remotely detectable element comprises a magnetic element.

21. A method of controlling a flow of cerebral spinal fluid, comprising the steps of:
implanting a cerebral spinal fluid flow device adapted for use with cerebral spinal fluid, said cerebral spinal fluid flow device having an inlet for said cerebral spinal fluid, an outlet for said cerebral spinal fluid and a first interior cavity fluidly coupled with said inlet and said outlet, and a first rotational element mounted in said first interior cavity in a pathway between said inlet and said outlet; and
providing resistance to rotation of said first rotational element to flow of said cerebral spinal fluid from said inlet to said outlet.

22. A method as in claim 21 wherein said first rotational element comprises a pinwheel mounted in said first interior cavity.

23. A method as in claim 22 wherein said providing step comprises braking said pinwheel to provide said resistance to rotation.

24. A method as in claim 23 wherein said providing step is provided by a pre-loaded spring mounted against said pinwheel to provide braking.

25. A method as in claim 24 wherein said resistance to rotation is provided radially with respect to said pinwheel.

26. A method as in claim 21 wherein said resistance to rotation is adjustable.

27. A method as in claim 21 wherein said resistance to rotation is provided by said body having a second interior cavity, a second rotational element mounted in said second interior cavity; and a viscous fluid contained in said second interior cavity providing rotational resistance to said second rotational element, wherein said second rotational element and said first rotational element are rotationally coupled.

28. A method as in claim 27 wherein said second rotational element is mounted co-axially with said first rotational element.

29. A method as in claim 27 wherein said second rotational element comprises a pinwheel.

30. A method as in claim 27 wherein said viscous fluid has a shear that increases with a rotational speed of said second rotational element.

31. A method as in claim 27 wherein said viscous fluid comprises silicone fluid.

32. A method as in claim 21 further comprising the step of a remotely detecting a remotely detectable element mounted with respect to said first rotational element.

33. A method as in claim 32 wherein said step of remotely detecting comprises detecting information regarding flow of said cerebrospinal fluid.

34. A method as in claim 32 wherein said step of remotely detecting comprises detecting information regarding a flow rate of said cerebrospinal fluid.

35. A method as in claim 33 wherein said step of remotely detecting comprises detecting information regarding a rotational speed of said first rotational element.

36. A method as in claim 32 wherein said remotely detectable element comprises a magnetic element.

37. An implantable cerebral spinal fluid flow device adapted for use with cerebral spinal fluid, comprising:
a body having an inlet for said cerebral spinal fluid, an outlet for said cerebral spinal fluid and a interior cavity fluidly coupled with said inlet and said outlet; and
rotational means associated with said interior cavity for providing resistance to flow of said cerebral spinal fluid from said inlet to said outlet.
